(19) United States
(12) Patent Application Publication
(10) Pub. No.: US 2009/0125322 A9
(43) Pub. Date: May 14, 2009

(54) Integrated virtual consultant

(75) Inventors: Michael Dahlin, Austin, TX (US); Eric Wohl, Austin, TX (US); Randolph Lipscher, Austin, TX (US)

Correspondence Address: LARSON NEWMAN ABEL & POLANSKY, LLP, 5914 WEST COURTYARD DRIVE, SUITE 200, AUSTIN, TX, 78730, US

(73) Assignee: RECARE, Inc.

(21) Appl. No.: 10/723,251

(22) Filed: Nov. 26, 2003

Related U.S. Application Data
(63) Continuation-in-part of application No. 09/992,035, filed on Nov. 23, 2001.
(60) Provisional application No. 60/252,820, filed on Nov. 22, 2000.

(51) Int. Cl. G06F 17/60

(52) U.S. Cl. 705/2; 705/1; 705/7

(57) ABSTRACT

In one particular embodiment, the disclosure is directed to method of providing a workflow interface. The method includes receiving template data associated with a medical workflow step, receiving automated decision support data associated with the medical workflow step, integrating the template data and the automated decision support data into an interface page associated with the medical workflow step, and initiating presentation of the interface page on a display of an electronic device.

### CROSS-REFERENCE TO RELATED APPLICATION(S)

The present application claims priority from U.S. provisional patent application No. 60/430,250, filed Dec. 2, 2002, entitled “Integrated virtual consultant,” naming inventors Michael Dahlin, Eric Wohl and Randolph Lipscher, which application is incorporated by reference herein in its entirety.

The present application claims priority from U.S. provisional patent application No. 60/430,451, filed Dec. 3, 2002, entitled “Integrated virtual consultant,” naming inventors Michael Dahlin, Eric Wohl and Randolph Lipscher, which application is incorporated by reference herein in its entirety.

### TECHNICAL FIELD

This disclosure relates in general to integrating advice into a workflow. More specifically, the invention relates to a method and system for integrating advice and virtual consultation into a workflow.

### BACKGROUND

Electronic Medical Records (EMRs) may improve the efficiency of medical workers and improve the quality of patient care.

Beginning in the 1970's, researchers have worked to provide clinical decision support systems to suggest likely diagnoses and to suggest useful treatments and/or tests. Such systems have not been widely adopted. Conventionally, a user would sit down at a desktop computer or terminal, enter findings, and view the system's suggestions. This mode of operation may not fit with traditional doctor-patient interactions.

As such, many typical systems suffer from deficiencies in providing virtual consultation. Many other problems and disadvantages of the prior art will become apparent to one skilled in the art after comparing such prior art with the present invention as described herein.

### DESCRIPTION OF THE DRAWINGS

FIGS. 1A and 1B show an exemplary integration of decision support functionality with an HCP EMR workflow.

FIG. 2 depicts an exemplary interface as displayed by the system.

FIG. 3 depicts an exemplary architecture of the system.

FIGS. 4, 5 , 6, 7, and 8 depict exemplary interfaces as displayed by the system.

FIG. 9 depicts an exemplary set of question for use in one exemplary embodiment of the system.

FIG. 10 depicts another exemplary embodiment of a system architecture.

### DETAILED DESCRIPTION

Electronic Medical Records (EMRs) may improve the efficiency of medical workers and improve the quality of patient care by allowing medical workers or patients or both to record, access, and analyze medical information and issue treatment orders. Medical information includes such data as patient history information, past medical records, reference information, patient symptoms, physical exam findings, laboratory orders, and medication orders.

At each stage of the visit, an electronic chart can present questions and alternatives to the patient, the nurse, and the physician about the patient's medical history, symptoms, diagnoses, and treatments. Such electronic interfaces provide standardization, accuracy, and access. Terminology is uniform, the choices are unambiguous and omissions unlikely, and the information is available to everyone. In addition, if properly implemented, such a system may improve efficiency by streamlining data gathering, although the success of such systems in improving efficiency in practice has been mixed.

Physicians often resist systems where the electronic expert usurps medical decision-making. Another approach would be to dynamically generate each screen to populate it with the most clinically relevant questions (based on the decision support system's output), but such a fully dynamic approach risks confusing users because screens are seldom the same. Conversely, if the expert advice is not closely integrated into the traditional workflow and requires significant extra effort to use, it will go unused. For example, historically expert systems that were packaged as a separate tool from the electronic medical record (EMR) have seen little use. In another current system, Medcin™, users must explicitly request that the system generate a decision support suggested questions or predicted answers in an extra step in the workflow. The Medicn™ approach is extra work for the user and risks not displaying important information if the user omits this step.

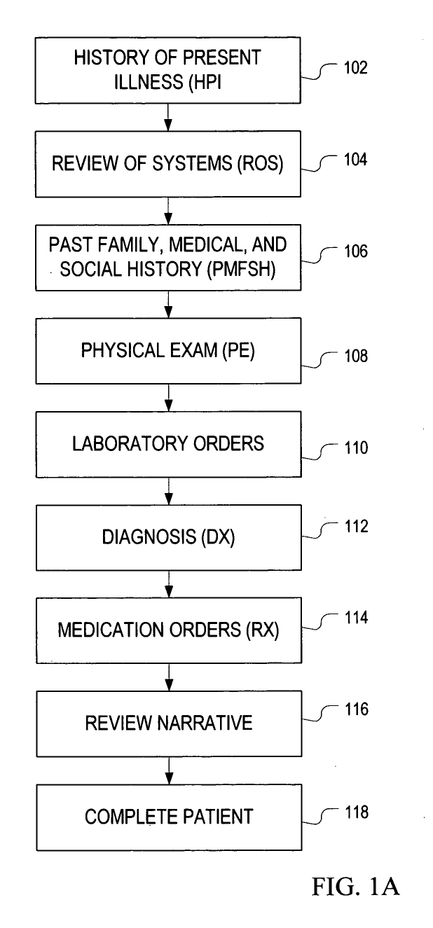

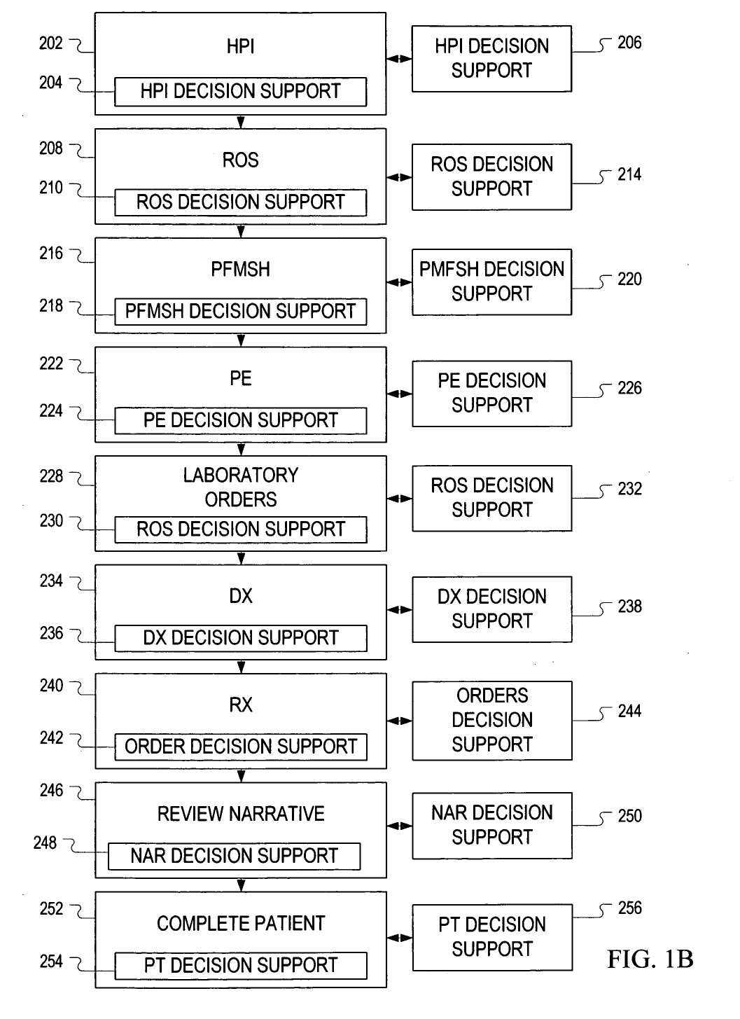

FIGS. 1A and 1B show an exemplary integration of decision support functionality with an HCP EMR workflow. The traditional workflow consists of several tasks, such as History of Present Illness (HPI) 102, Review of Systems (ROS) 104, Past family medical and social history (PMFSH) 106, Physical Exam 108, Laboratory orders 110, Diagnosis (Dx) 112, Medication orders (Rx) 114, review narrative 116, and complete patient 118. Tasks may be added, removed, skipped, combined, or split without changing the basic nature of this flow. The arrows in the figures illustrate a common task order, although systems may allow other orders or allow random access between tasks.

The extended workflow adds the ability to view and interact decision support information regarding the current patient by (a) providing the decision support information associated with each traditional task while some or all traditional tasks are being performed and/or (b) providing a “context sensitive” link from each task to the associated decision support information.

In a particular embodiment, the system integrates decision support information with a template-based electronic medical records (EMR) system by simultaneously displaying the template information and related decision support information. Some embodiments will simultaneously display template information, findings information, and related decision support information.

Template information is information that prompts or enables the user to enter findings information and is selected for display based on criteria including the patient's chief complaint (e.g. “chest pain” or “sore throat” ), or the current task (e.g. “history of present illness” or “selected diagnosis”), or both. Template information to be displayed may also be selected based on factors such as demographic information about the patient, clinic, physician specialty, or physician preferences.

Findings information includes information about the current patient. Examples of such information include complaint onset, complaint duration, complaint quality, complaint severity, causes of complaint, relievers of complaint, review of systems, physical condition, history, active problems, past problems, test results, current medications, demographic information, diagnosis, and prescribed medications. In an exemplary embodiment, this information is encoded so that each finding is associated with a unique identifier in a medical nomenclature framework. In another embodiment, findings are encoded as Booleans (representing present/not present for example), tri-state (present/not present/no-comment, for example), integer values, and character strings.

Decision support information is information generated by a decision support system based on input information that may include finding information. Examples of decision support systems include neural networks, Bayesian networks, expert systems, and decision trees. The output comprises input fields or output data or both. In one exemplary embodiment, input fields allow at least one of (a) entry of findings or (b) entry of orders (e.g. prescription orders or lab tests). In another embodiment, output fields include at least one of (a) warnings or alerts relating to the treatment of the patient, (b) recommendations relating to the treatment of the patient, and (c) information relating to the treatment of the patient.

FIG. 1B depicts the decision support information integrated with the workflow. In this exemplary embodiment, the HPI decision support information 204 provided by an HPI decision support system 206 is integrated with an HPI interface 202. Similarly, ROS decision support information 210 provided by ROS decision support system 214 is integrated with ROS interface 208, PMFSH decision support information 218 provided by PMFSH decision support system 220 is integrated with PFMSH interface 216, PE decision support information 224 provided by PE decision support system 226 is integrated with PE interface 222, ROS decision support information 230 provided by ROS decision support system 232 is integrated with Laboratory Orders 228, DX decision support information 236 provided by DX decision support system 238 is integrated with DX interface 234, Orders decision support information 242 provided by Orders decision support system is integrated with RX interface 240, Narrative decision support information 248 provided by Narrative decision support system 250 is integrated with Review Narrative interface 246, and Patient decision support information 254 provided by Patient decision support system 256 is integrated with Complete Patient 252.

Different embodiments integrate decision support information with template information in different ways, including: (a) the system highlights options corresponding to suggestions for treatment or tests in the available options in the workflow, (b) the system displays suggestions of what the physician should do in a particular situation in the workflow, (c) the system integrates requests for information or suggestions for treatment into the menu of questions/actions available to the physician at a specific point in the workflow, and/or (d) the system displays an icon alerting the HCP that more information is available or that a possible error (such as prescribing a contra-indicated medication or test) is being made, among others.

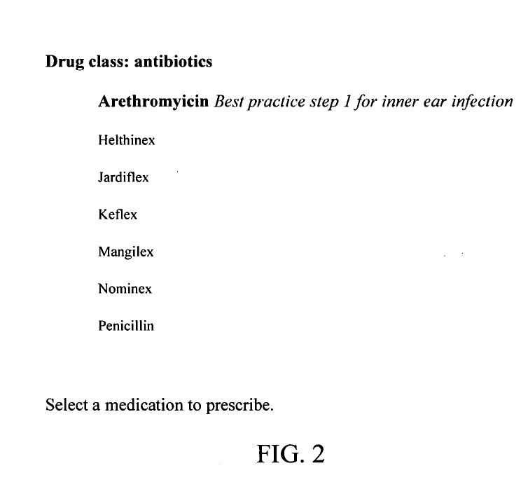

FIG. 2 illustrates one embodiment of a strategy of displaying suggestions. In this example, while a physician is using his workflow system to write an electronic prescription for a medication to treat a specific condition, the system highlights the decision support system's suggestion for the best medication to use based on the patient's current condition and past medical records and previous treatments. In this example, since step 1 therapy has not been tried yet, that therapy is highlighted. Similar techniques can be used to provide relevant information in other steps of the workflow. Highlighting may, for example, be accomplished by (a) changing the font, size, and/or color of highlighted text, (b) adding an icon near the highlighted element, or (c) adding additional text or graphical information near the highlighted element.

The highlighting technique can be used in several ways. This approach allows the system to offer suggestions but the final decision-making rests with the health care provider (HCP). This is beneficial for both legal reasons (the computer system does not make automatic selections, all selections are made by a licensed professional) and for acceptance reasons (HCPs may not desire that computers take over decision making.)

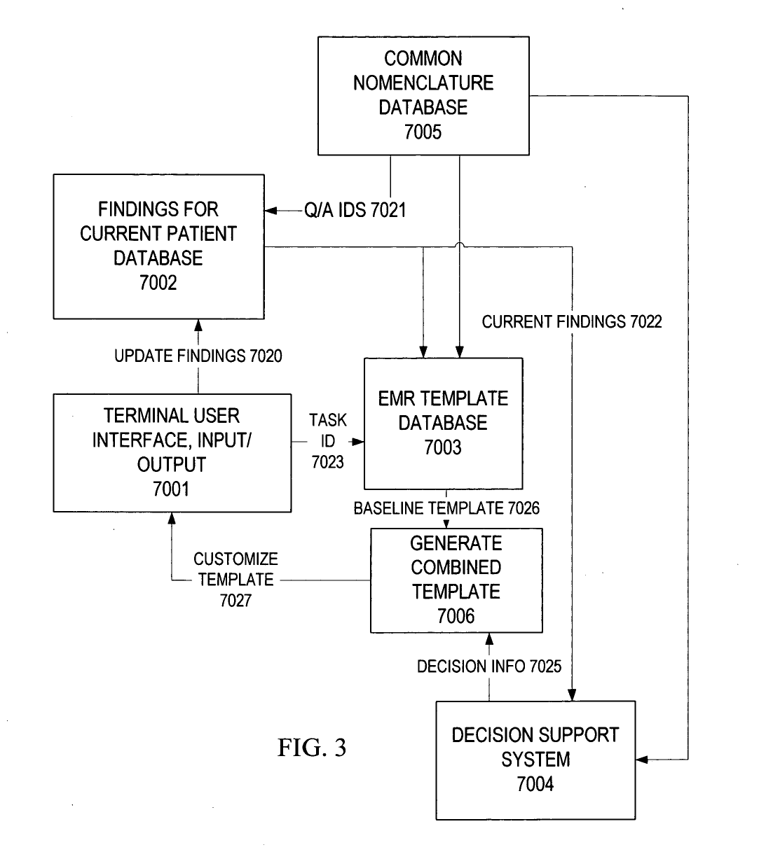

The numbers below refer to FIG. 3, described later in this description. In the highlight order embodiment, the system highlights suggested orders (e.g., prescription to write, lab test to order). First, using the terminal 7001, the HCP activates an order task (e.g., the Rx task or the lab order task). Second, using the selected task 7023 and (optionally) the current findings for the patient 7022 as input, the system retrieves from the EMR template database a baseline template 7026 comprising a standard list of orders. This list may be a standard list of common orders, a per-HCP “hotlist”, a list of standard orders, common orders for the patient's chief complaint, a list of all available orders, a hierarchically navigated or searched list of all available orders, or the like. The decision support system 7004 generates a list of predicted orders 7025 based on the current findings 7022. The generate combined template module 7006 identifies any orders that appear both on the baseline template 7026 and the predicted set of orders 7025 and generates a new customized template 7027 where the matching elements are highlighted.

In one embodiment, the customized template is an HTML form where the matching elements are highlighted with a distinctive font, font size, or color or with an icon. In another exemplary embodiment, the customized template is an XML form where the matching elements type is changed from a standard “ORDER” to a type of “HIGHLIGHTED_ORDER”, that the user interface module has been programmed to render in a distinctive manner such as a different font, size or color from an ORDER. Optionally, as more information is entered into the terminal 7001 and stored in the findings for current patient database 7002, if the set of orders output by the decision support system 7004 changes from what is displayed, the system may dynamically alter the user display by highlighting the updated set of matching items.

In the highlight answers embodiment, the system highlights predicted answers to displayed questions; (1) using the terminal 7001 the HCP activates a data input task (e.g., HPI, ROS, Physical Exam, PMFSH) and (2) using the selected task 7023 and (optionally) the current findings for the patient 7022 as input, the system retrieves from the EMR template database a baseline template 7026 comprising a standard list of questions and selectable answers. This list may be a standard list of common questions and answers, a per-HCP “hotlist” of questions and answers for the task, a list of questions and answers for the task, common questions and answers for the patient's chief complaint and current task, a list of all available questions and answers for the current task, a hierarchically navigated or searched list of all available questions and answers for the current task, or the like. The decision support system 7004 generates a list of predicted answers 7025 based on the current findings 7022. Note that this set of may correspond to a subset of the questions for baseline template. In particular, this set may comprise predicted answers to the most diagnostically relevant questions given the current findings 7022). The generate combined template module 7006 identifies answers that appear both on the baseline template 7026 and the predicted set of answers 7025 and generates a new customized template 7027 where the matching elements are highlighted. In one exemplary embodiment, the customized template is an HTML form where the matching elements are highlighted with a distinctive font, font size, or color or with an icon. In another exemplary embodiment, the customized template is an XML form where the matching elements type is changed from a standard “ANSWER” to a type of “HIGHLIGHTED_ANSWER”, that the user interface module has been programmed to render in a distinctive manner such as a different font, size or color from an ANSWER. Optionally, as more information is entered into the terminal 7001 and stored in the findings for current patient database 7002, if the set of answers output by the decision support system 7004 changes from what is displayed, the system may dynamically alter the user display by highlighting the updated set of matching items.

In the highlight parameters embodiment, the system highlights predicted parameters to an order being issued. For example, in this embodiment, the system predicts which parameters for a medication are likely to be selected and highlights them. This makes it easy for the HCP to quickly prescribe the most appropriate form (e.g., tablet, IV, suppository), dosage, frequency, and so forth) for the current patient given the findings for the current patient. Using the terminal 7001, the HCP activates an order parameter input task (e.g., write prescription, order x-ray, order blood test). Using the selected task 7023 and (optionally) the current findings for the patient 7022 as input, the system retrieves from the EMR template database a baseline template 7026 comprising a standard list of parameters for the order. The decision support system 7004 generates a list of predicted parameter selections 7025 based on the current findings 7022 and the current task 7023. The generate combined template module 7006 identifies parameters that appear both on the baseline template 7026 and the predicted set of parameters 7025 and generates a new customized template 7027 where the matching elements are highlighted. In one embodiment, the customized template is an HTML form where the matching elements are highlighted with a distinctive font, font size, or color or with an icon, among others. In one exemplary embodiment, the customized template is an XML form where the matching elements type is changed from a standard “PARAMETER” to a type of “HIGHLIGHTED_PARAMETER”, that the user interface module has been programmed to render in a distinctive manner such as a different font, size or color from a PARAMETER. Optionally, as more information is entered into the terminal 7001 and stored in the findings for current patient database 7002, if the set of answers output by the decision support system 7004 changes from what is displayed, the system may dynamically alter the user display by highlighting the updated set of matching items.

In the highlight questions embodiment, the system highlights questions that may be diagnostically relevant to the current patient. Using the terminal 7001 the HCP activates a data input task (e.g., HPI, ROS, PMFSH, Physical exam). Using the selected task 7023 and (optionally) the current findings for the patient 7022 as input, the system retrieves from the EMR template database a baseline template 7026 comprising a standard list of questions to ask. The decision support system 7004 generates a list of diagnostically most relevant questions 7025 based on the current findings 7022. The generate combined template module 7006 identifies questions that appear both on the baseline template 7026 and the important set of questions 7025 and generates a new customized template 7027 where the matching elements are highlighted. In one exemplary embodiment, the customized template is an HTML form where the matching elements are highlighted with a distinctive font, font size, or color or with an icon. In another exemplary embodiment, the customized template is an XML form where the matching elements type is changed from a standard “QUESTION” to a type of “HIGHLIGHTED_QUESTION”, that the user interface module has been programmed to render in a distinctive manner such as a different font, size or color from a QUESTION. Optionally, as more information is entered into the terminal 7001 and stored in the findings for current patient database 7002, if the set of answers output by the decision support system 7004 changes from what is displayed, the system may dynamically alter the user display by highlighting the updated set of matching items.

In the add information embodiment, the system highlights questions, orders, answers, or parameters, among others (collectively, “elements”), that may be relevant to the current patient by adding additional information about the element. Using the terminal 7001, the HCP activates a data task. Using the selected task 7023 and (optionally) the current findings for the patient 7022 as input, the system retrieves from the EMR template database a baseline template 7026 comprising a standard list of elements to display. The decision support system 7004 generates a list of diagnostically relevant elements 7025 based on the current findings 7022. The decision support system 7004 further generates for zero or more generated elements an “additional information” field. This field may be an explanation or comment on the rule that generated the element. For example, in a rules based system manually generated by experts, the experts may associate explanatory matter or reference matter with specific rules. For example, in a neural nets based system, the system typically generates “confidence” estimates indicating how likely a particular prediction is to be correct based on past experience. The generate combined template module 7006 identifies elements that appear both on the baseline template 7026 and the generated set of elements 7025 and generates a new customized template 7027 where the matching elements are highlighted by adding the “additional information” to the matching elements. In one embodiment, the customized template is an HTML form where the matching elements are highlighted by adding the specified data as additional text near the original element. In another exemplary embodiment, the customized template is an XML form where the matching elements type is of type “ELEMENT” with an attribute field “ELEMENT_ATTRIBUTE” that is set to the value of the “additional information” where that the user interface module has been programmed to render ELEMENT_ATTRIBUTES in a distinctive manner near its ELEMENT. Optionally, as more information is entered into the terminal 7001 and stored in the findings for current patient database 7002, if the set of answers output by the decision support system 7004 changes from what is displayed, the system may dynamically alter the user display by highlighting the updated set of matching items.

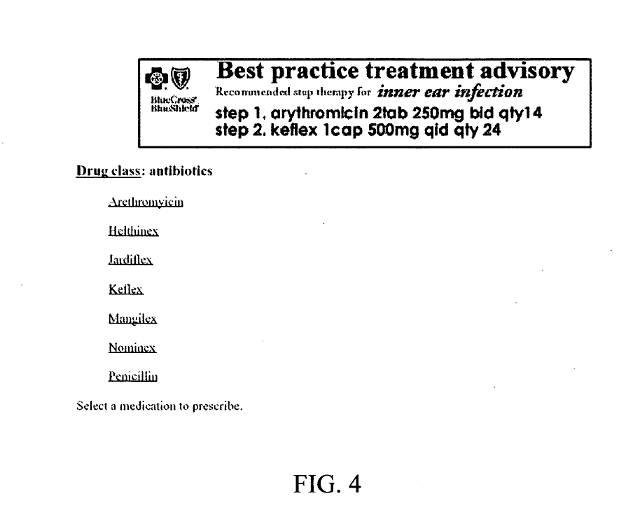

FIG. 4 also illustrates the strategy of displaying suggestions. In this example, while a physician is using his workflow system to write an electronic prescription for a medication to treat a specific condition, the system provides the decision support system's suggestion for the best medication to use based on the patient's current condition and past medical records. Similar displays may be included for other steps in the workflow: suggestions for important questions to ask during the history of present illness or review of systems, suggestions for examinations to undertake during the physical exam, and suggestions for laboratory tests to order on lab order screens. The displays may be informational only, or they may be “active” controls that the user may manipulate to input data into the EMR, among others.

This technique can be applied in a add information embodiment, add questions embodiment, a add orders embodiment, add answers embodiment similar to the highlight questions, highlight orders, and highlight answers embodiments detailed above. The difference is that rather than highlighting elements that appear both on the baseline template 7026 and the predicted/suggested elements 7025, the system generates a new template comprising the union of elements that appear in both lists.

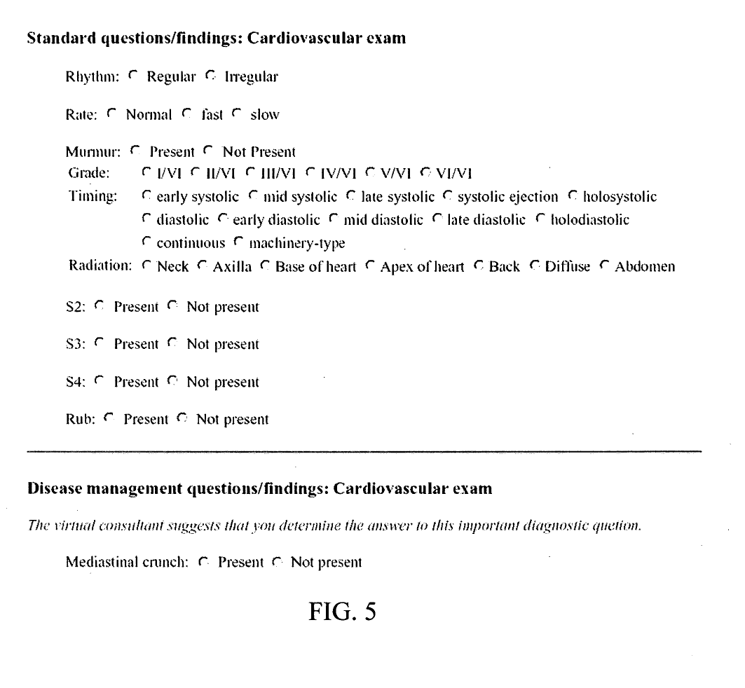

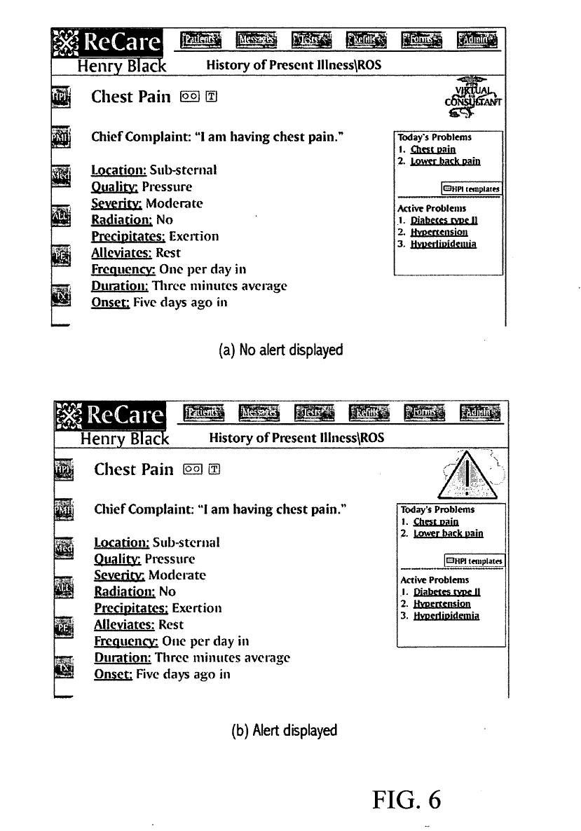

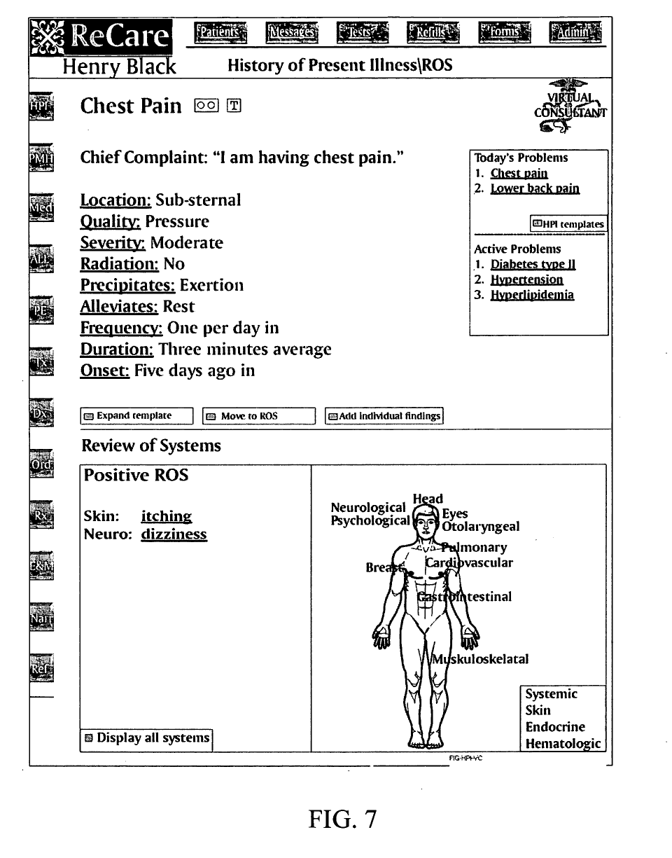

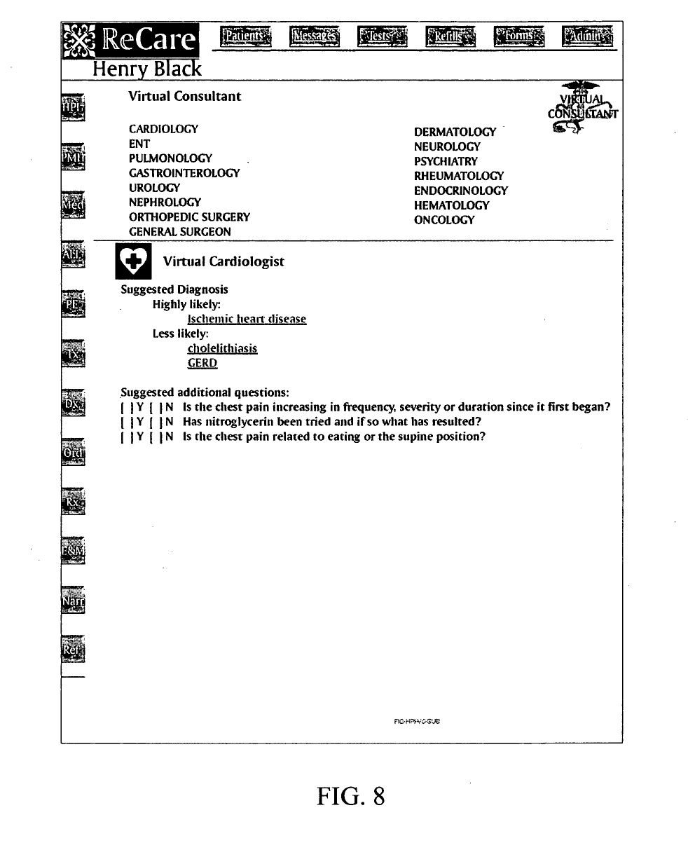

FIG. 5 illustrates the strategy of integrating requests for information or suggestions for treatment into the menu of questions/actions available to the physician at a specific point in the workflow. In this example, physical exam questions specified by the decision support system have been added to the standard physical exam template for the patient's condition. This prompts the HCP to ask questions he might not commonly ask that the decision support system believes are important for managing the patient's condition effectively. Similar techniques may be used to provide relevant information in other steps of the workflow.

This technique may be applied to other templates such as in an add questions embodiment, an add orders embodiment, an add answers embodiment similar to the highlight questions, highlight orders, and highlight answers embodiments detailed above, among others. Rather than highlighting elements that appear both on the baseline template 7026 and the predicted/suggested elements 7025, the system generates a new template comprising the union of elements that appear in both lists.

An attribute of the system is that any newly generated question, order, answer, or parameter elements may be pre-populated with answers already stored in the findings for current patient database 7002. This eliminates manually re-entering the same information that is found in current systems where the decision support module is not integrated with the EMR. Also, as new data are entered into the current patient database 7002, the set of medically relevant elements may change. For example, one “branch” of a path of inquiry may become less relevant when a question is answered. In that case, the system allows the display to be regenerated to eliminate the irrelevant questions based on the new information.

FIG. 6 illustrates the strategy in which the system displays an icon alerting the HCP that more information is available or that a possible error is being made. In this example, the system detects that there is information that the physician may wish to see. Although the system does not display the information, it notifies the physician with a visual, auditory or tactile cue. The physician may then activate a subroutine to display the information associated with that alert. Examples of such alerts include: the system has identified a crucial question(s) to ask for the HPI or ROS; or the system has identified a likely test, order, or medication to treat or diagnose the patient; the system has detected that a medication being prescribed or a test being ordered is contraindicated, non-formulary, interacts with other medications, may cause allergic reactions with the patient, or is otherwise not medically indicated or optimal.

Combinations or variations of these strategies may also be used. For example, the functionality of FIG. 4 and FIG. 5 can be combined: the system might display suggestions for tests to run or medications to prescribe in a manner similar to FIG. 4 but these displays may be “active” and allow input of findings or orders as in FIG. 5.

In addition to integrating specific decision support information in the specific stages of the EMR workflow, the system provides a “context sensitive” link from each stage the workflow to the relevant decision support information.

FIG. 7 illustrates an EMR history present illness screen. In the upper right hand corner is an icon that may be activated to bring up the “virtual consultant” decision support information. Similar icons appear on the other major screens of the EMR (PMFSH, physical exam, orders, Rx, etc.).

As FIG. 8 illustrates, if the decision support icon is activated from the history present illness screen, the system displays context sensitive decision support information. In the case history present illness, the system displays a list of questions that are like to be useful in forming differential diagnoses.

Whether the technique of integrating decision support information into the regular screen or the technique of providing the link to such information is used, the information may be displayed if the information is relevant to the current step in the EMR workflow.

The following illustrates examples of the types of information that are relevant difference stages of the workflow:

Workflow stage:

HPI/ROS: additional questions that should be asked; likely diagnoses; highlight important findings;
PMFSH: additional questions that should be asked; highlight important findings;
physical exam: findings that should be examined; highlight important findings;
lab/radiology orders/results: recommended tests; criteria for ordering it asked; contraindications for a test; economics/formulary requirements for a test; flight/highlight normal/abnormal values; detect unusual trends;
Rx: recommended medication; contraindications; interaction; allergies; warning; formulary information; step therapy recommendations; best practice recommendations.

In addition to enhancing the EMR workflow to accommodate decision support, this system enhances the decision support system by integrating the information available from the EMR workflow to generate precise and relevant recommendations without requiring the HCP to re-enter data.

The particular technique used to implement the decision support system or generate the decision support information may take various forms. Examples of such systems include expert system rules, neural networks, handcrafted rules, Boolean rules, Bayesian rules, decision trees, and inductive logic programming.

Decision support information is generally of two types. First, the information they reflect is generic “reference material” associated with a particular step in the workflow or with a particular template used in the workflow, for example, “likely medications for patients complaining of chest pain.” Second, the information they reflect is the output of the decision support rules that consider factors, such as the patient's Chief complaint, past findings, current findings, patient demographics, patient formulary, patient PMFSH, other medications the patient is taking, and the physician specialty. This second type of decision support information is extracted directly from the EMR and sent to the decision support system without the HCP manually re-entering or transferring the information. Thus, decision support information may be continuously updated as additional findings about the current patient are entered. In addition, when the user displays one type of decision support information—decision support algorithms—this display is customized to reflect what is known about the current patient.

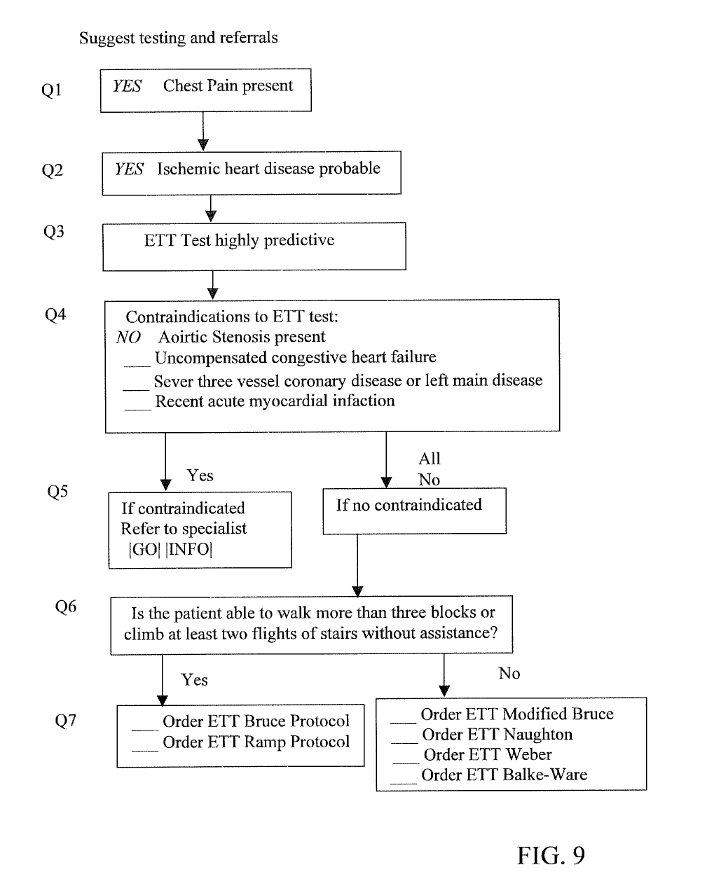

For example, consider an HCP who is using the EMR system to select tests for the current patient who has complained of chest pain. In earlier steps (the HPI/ROS and Physical exam), the HCP noted in the medical record that Aortic Stenosis is not present in the patient, but the HCP has not made any notation about whether uncompensated congenital heart failure, severe three vessel coronary disease or left main disease, recent acute myocardial infarction, are present or not present for the patient, nor has the physician indicated the patient's exercise capability in the record. In an exemplary implementation, while viewing the “select tests and referrals” task in the EMR, HCP selects “Virtual consult”, and, since the active EMR task is “select tests and referrals”, the system displays a list of “suggested testing and referrals” as shown in FIG. 9. Note that this display is initialized to include values based on both what has been explicitly entered into the EMR (That chest pain is present and Aortic Stenosis is not present) and what has been inferred by the decision support system based on findings in the EMR (that Ischemic heart disease is probable for this patient based on the current set of findings.)

As additional information is entered, the display of the algorithm is updated accordingly. For example, in an exemplary implementation, if in Q4 the HCP marks “YES” for uncompensated congestive heart failure present, the “all NO” path would disappear from the screen. For example, in an exemplary implementation, if the HCP changes the element Q2 “Ischemic heart disease probable” from “YES” to “NO” (thus using his judgment to overrule the decision support systems inference from the findings in the EMR), the system would replace the lines Q4, Q5, Q6, Q7, Q8 that describe the algorithm for selecting an ETT protocol with new lines describing the algorithm for selecting a test that is appropriate when Ischemic heart disease is not considered highly probable such as Thallium myocardial scan. If the HCP changes element Q2 from “YES” to “Blank/no finding”, the system would expand the algorithm to show both paths with the appropriate decision criteria listed (as is done for several later questions in this example.)

Furthermore, as additional information is entered, the corresponding findings in the EMR are updated. For example, if the HCP notes that Uncompensated congestive heart failure is present (or not present) in step Q4, the EMR findings are so updated.

If the doctor selects an order (for a medication, test, procedure, etc.), the system may change the active task for the EMR to be the task for ordering the selected item. For example, if a displayed algorithm ends up recommending that the doctor prescribe a beta blocker, and the doctor selects that option, the system may display the EMR system with the “Rx” task active and the electronic prescription pad initialized to display beta blocker with recommended doses highlighted. The HCP would then fill in any remaining parameters and continue with the EMR process.

Each element in the algorithm may optionally be associated with additional information that the doctor may view. This information may describe the medical reasoning behind that step in the algorithm. For example, if “info” is selected for Q2 in the FIG. 9, the system could display the list of findings that indicate to the decision support system that Ischemic heart disease is probably along with an estimated probability that that is the correct clinical diagnosis and a list of other likely diagnoses and their probabilities. For example, if “info” is selected in Q3 in FIG. 9, the system would display the medical reason that ETT is the best test to use in the specified situation. For example, such a message might read:
“In this patient with a high predicted value of a diagnosis of ischemic heart disease, an ETT has a 98% sensitivity and a 90% specificity. Thallium scans are more costly. Thallium scans are more useful in patients with a lower predictive value of ischemic disease than this patient. ETT approaches the sensitivity and specificity of a thallium scan in this patient. Therefore ETT is a more cost effective alternative to a Thallium scan for this patient.”

In addition to algorithms for selecting tests, similar algorithms are used for selecting medications (e.g., step therapy algorithms to select cost effective first-line medications for conditions before resorting to more expensive second-line medications), for conducting physical exams or reviews of systems, or history of present illness questioning, and for selecting diagnoses.

In addition to a separate display of an algorithm, the same functionality may be integrated into the steps of the EMR workflow using combinations of the types of techniques described above. For example, the questions Q4 and Q6 in FIG. 9 might be added to the standard history of present illness questions (e.g., using the technique described in FIG. 5). When the “Order tests” task is active, if these questions were all answered appropriately and the decision support system believes ETT to be indicated and the physician has entered a finding of “able to walk more than three blocks and/or climb stairs without assistance”, the system would highlight the “ETT Bruce Protocol” and “ETT Ramp Protocol” on the list of available labs (e.g., using the technique described in FIG. 2). Or, if the doctor chose to skip answering one or more of the Q4 questions and the doctor selects “ETT Bruce Protocol” as a lab, the system would display a warning such as the one shown in FIG. 6. Alternately, if the doctor entered YES for one of the Q4 questions and the doctor selects “ETT Bruce Protocol” as a lab, the system would display a more insistent warning such as a dialog box explaining the contraindication and asking the doctor to confirm the order.

FIG. 3 illustrates the components in an exemplary realization. In this realization, a user interacts with a Terminal/user interface module 7001. This module displays information to the user and receives user input. In one exemplary embodiment, this module comprises a HTTP/HTML browser such as Netscape Navigator or Microsoft Internet Explorer running on an operating system such as Solaris, Linux, or Microsoft NT and a computer such as a National Semiconductor WebPad demonstration unit, S3 Web Pad, Dell Dimension 5100, or Sony Vaio. The remaining modules may run on the same computer as the user interface module, or they may run on a separate computer that communicates with the user interface computer via a wired or wireless network link.

The current patient database 7002 stores data about the current patient including findings, demographic information, and patient ID, among others. This information includes both data entered during the current encounter and data entered during past encounters as well as data retrieved from external data sources such as legacy databases.

The EMR template database 7003 stores medical information representing sets of questions to ask or orders to issue during a medical encounter. In an exemplary realization, this information is organized into templates of related questions. For example, related questions may include the set of questions to ask for a patient whose chief complaint is “chest pain.” In this realization, each template is further organized into tasks where each task represents a subset of questions that are displayed together. For example, in a typical EMR one task might be “gather history of present illness information”, another might be “conduct physical exam”, and another might be “write prescriptions.”

The decision support system 7004 takes as input the current set of data and findings about a patient and produces as output (a) additional questions to ask about the patient (e.g., a differential diagnosis question to ask), (b) predictions to the answers to questions about the patient (e.g., predict a likely diagnosis or predict a likely medication to prescribe), or templates consisting of zero or more questions, zero or more predictions, and (optionally) Boolean logic for the conditions under which the questions should be asked or predictions made. We refer to the output of the decision support system collectively as “predicted elements”.

The decision support system 7004 may comprise rules based decision support rules. The decision support system may also comprise a learning-based predictor.

In this realization, the patient information database 7002, EMR template database 7003, and decision support database 7004 use a common nomenclature 7005 for identifying questions, answers, orders, and parameters. That is questions, answers to a particular question, order types, and parameters (to an order or question) are identified with a unique identifier. This unique identifier is common among these three modules. This correspondence may be implemented directly—each module may use the same internal identifiers for the same elements—or it may be implemented via translation software that maintains a database of elements and IDs for those elements in each module and can map between them. Note that the overall common nomenclature may be realized by several nomenclatures where each is responsible for a different subsets of the elements. For example, the ICD9 nomenclature is a standard nomenclature for diagnoses.

When the user interface software wishes to change the display (e.g., in response to user input), it sends user input containing updates to findings 7020 to the findings for current patient database 7002 and it sends a request to display the next set of information to the EMR template database. The generated combined template 7006 may then deliver the display or display data.

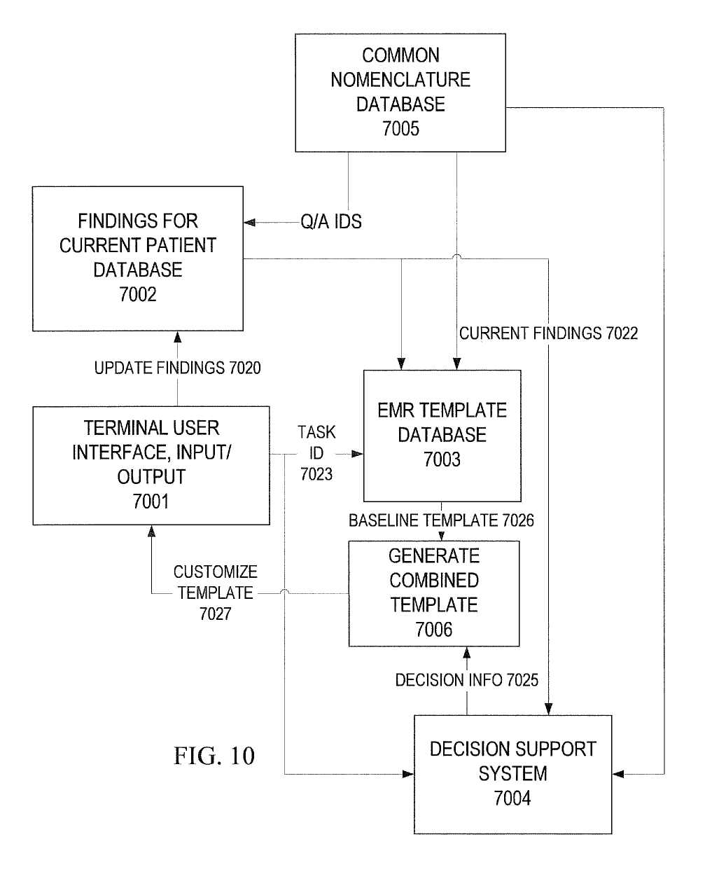

In another realization, the decision support system takes as input the current task and generates predicted answers and suggested questions for that task. This realization is illustrated in FIG. 10.

In another embodiment, the generate combined template 7006 and the terminal 7001 are combined into a single module that takes a baseline template 7026 and a predicted set of answers or suggested questions 7025 and combines them and displays them to the user.

In one exemplary embodiment, the system includes a medical electronic patient record workflow system and a medical decision support system in which decision support information is integrated into the electronic patient record workflow or electronic patient record information is integrated into the decision support process. The system may further include two or more separate tasks in the medical electronic patient record workflow and display different subsets of categories of decision support information according to the current active task in the medical electronic patient record workflow. The system may further include decision support information associated with a specific task in the electronic patient record workflow displayed along with information from the electronic patient record workflow. The system may further include decision support information associated with a specific step in the electronic patient record workflow displayed by triggering an action while that step in the electronic patient record workflow is active. In addition, decision support information may be displayed using a plurality of methods including: an icon indicating an alert that additional information is available, a list of questions that are likely to be useful, a list of lab, radiology, staff, therapy, or medication orders that are likely to be useful, or an alert that an action being taken may have adverse consequences. In another embodiment, data may be entered into the electronic medical record system and directly provided to the decision support logic. The decision support logic may select relevant information based on electronic medical record findings about the current patient. Decision algorithms may be customized based on findings in the current patient's electronic medical record when displayed. Decision algorithms may be repeatedly run as additional findings are entered into the electronic patient record system, thus refining the decision support information available to the user. Decision support information to be displayed may consist of elements selected as relevant based on the current active task in the EMR workflow and elements selected as relevant based on the current findings for the current patient. Decision support information may be displayed using a plurality of the following techniques: (a) the system displays suggestions of what the physician should do in a particular situation in the workflow, (b) the system highlights options corresponding to suggestions for treatment or tests in the available options in the workflow, (c) the system integrates requests for information or suggestions for treatment into the menu of questions/actions available to the physician at a specific point in the workflow, (d) the system displays an icon alerting the HCP that more information is available or that a possible error (such as prescribing a contra-indicated medication or test) is being made, and/or (e) the system provides an option for the HCP to explicitly activate and view decision support algorithms relevant to the current task and patient. The user may query the system for information about the medical reasons underlying decision support algorithms.

Other embodiments include a medical electronic patient record workflow system and a medical decision support system in which decision support algorithms are integrated into the electronic patient record workflow. Decision support algorithms may be displayed with choice data pre-populated from findings present in the electronic patient record. New findings or orders may be entered in the decision support algorithm display or existing findings or orders may be modified in the decision support algorithm display and those new or modified findings or orders added to or modified in the electronic patient record used by the electronic patient record workflow system. The findings questions or orders that the decision support algorithms select as relevant may be separated into questions or orders corresponding to electronic medical record tasks and the decision support elements may be displayed and updated in the course of the EMR workflow. Decision support algorithms may be displayed with branches of the algorithm display pruned to eliminate unlikely or undesired paths based on the current findings. The user may query the system for information about the medical reasons underlying decision support algorithms.

Further embodiments include a template-based electronic medical record (EMR) system. The system may be executed on a device such as a wireless tablet, palm-sized computer, or desktop computer by integrating decision support functionality with an EMR workflow.

An embodiment may include a system that integrates electronic medical records with decision support logic in a way that is convenient for physicians to use yet places full control of the system in physician hands. The system may include a consistent base template that doctors can become familiar with plus automatic integrated decision support suggestions. The system may further include a strategy that integrates decision support technology into the overall physician workflow in a consistent and useful way.

Further aspects of the system may be found in various embodiments described above, including: highlight order embodiment, highlight predicted answer embodiment, highlight question embodiment, highlight order parameter embodiment, add order embodiment, add predicted answer embodiment, add question embodiment, add order parameter embodiment, highlight by adding information embodiment. However, various embodiments may be envisaged.

The interface pages depicted in the figures and described above may be altered by rearranging elements, enhancing graphics, adding or supplementing multimedia elements, or including alternate text, controls, or elements. For example, the depicted interface pages may be enhanced with interactive graphic or pictorial elements.

The above disclosed subject matter is to be considered illustrative, and not restrictive, and the appended claims are intended to cover all such modifications, enhancements, and other embodiments that fall within the scope of the present invention. Thus, to the maximum extent allowed by law, the scope of the present invention is to be determined by the broadest permissible interpretation of the following claims and their equivalents, and shall not be restricted or limited by the forgoing detailed description.

What is claimed is:

1. A method of providing a workflow interface, the method comprising:
receiving template data associated with a medical workflow step;
receiving automated decision support data associated with the medical workflow step;
integrating the template data and the automated decision support data into an interface page associated with the medical workflow step; and
initiating presentation of the interface page on a display of an electronic device.

2. The method of claim 1, further comprising:
receiving patient finding data; and
basing selection of the automated decision support data at least partially on the patient finding data.

3. The method of claim 1, further comprising sorting the template data.

4. The method of claim 1, further comprising highlighting a portion of the template data.

5. The method of claim 1, further comprising annotating a portion of the template data.

6. The method of claim 1, further comprising initiating presentation of decision support text on the display.

7. The method of claim 1, further comprising adding additional template elements to the interface page.

8. The method of claim 1, wherein the medical workflow step comprises recording a history of present illness.

9. The method of claim 1, wherein the medical workflow step comprises a review of systems.

10. The method of claim 1, wherein the medical workflow step comprises a diagnosis.

11. The method of claim 1, wherein the medical workflow step comprises a prescription writing step.

12. A device configured to display a user interface associated with a step in a medical workflow, the user interface comprising template data integrated with automated decision support data, the template data and the automated decision support data being associated with the step in the medical workflow, the automated decision support data being based on medical finding data.

13. The device of claim 12, wherein the automated decision support data annotates a portion of the template data.

14. The device of claim 12, wherein the automated decision support data includes additional template elements.

15. The device of claim 12, wherein the automated decision support data functions to highlight a portion of the template data.

16. The device of claim 12, wherein the automated decision support data functions to sort at least a portion of the template data.

17. The device of claim 12, wherein the automated decision support data is associated with patient findings.

18. The device of claim 12, wherein the step in the medical workflow is associated with history of present illness.

19. The device of claim 12, wherein the step in the medical workflow is associated with review of systems.

20. The device of claim 12, wherein the step in the medical workflow is associated with diagnosis.

21. The device of claim 12, wherein the step in the medical workflow is associated with prescription preparation.

22. The device of claim 12, wherein the user interface device is a portable computational circuitry configured to communicate with a wireless network.

23. A system comprising:
a processor; and
a storage medium storing:
instructions operable to direct the processor to retrieve template data associated with a step in a medical workflow;
instructions operable to direct the processor to retrieve automated decision support data associated with the step in the medical workflow;
instructions operable to direct the processor to integrate the template data and the automated decision support data into an interface page associated with the step in the medical workflow; and
instructions operable to direct the processor to initiate presentation of the interface page to a user interface device.

24. The system of claim 23, further comprising:
instructions operable to direct the processor to retrieve patient finding data; and
instructions operable to direct the processor to identify the automated decision support data in response to the patient finding data.

25. The system of claim 23, wherein integrating the template data and the automated decision support data comprises sorting the template data.

26. The system of claim 23, wherein integrating the template data and the automated decision support data comprises highlighting a portion of the template data.

27. The system of claim 23, wherein integrating the template data and the automated decision support data comprises annotating a portion of the template data.

28. The system of claim 23, wherein integrating the template data and the automated decision support data comprises adding decision support text to the template data.

29. The system of claim 23, wherein integrating the template data and the automated decision support data comprises adding additional template elements.
